(19) United States
(12) Patent Application Publication
(10) Pub. No.: US 2016/0106091 A1
(43) Pub. Date: Apr. 21, 2016

(54) FREEZE PRESERVATION OF LIVING CELLS

(71) Applicant: UNIVERSITE DE NAMUR, Namur (BE)

(72) Inventors: Christophe MEUNIER, Floreffe (BE); Pierre VAN CUTSEM, Ottignies (BE); Bao-Lian SU, Saint-Servais (BE)

(73) Assignee: UNIVERSITE DE NAMUR, Namur (BE)

(21) Appl. No.: 14/896,882

(22) PCT Filed: Jun. 11, 2014
(86) PCT No.: PCT/EP2014/062109

§ 371 Date: Dec. 8, 2015

(30) Foreign Application Priority Data
Jun. 12, 2013 (EP) PCT/EP2013/062102

(51) Int. Cl. A01N 3/00; C12N 5/04

(52) U.S. Cl. CPC A01N 3/00; C12N 5/04

(57) ABSTRACT

A method is for the cryopreservation of a (plant) cell, (plant) organelle or (plant) tissue, where the (plant) cell, (plant) organelle or (plant) tissue is encapsulated in a matrix formed of an abiotic material in presence of one or more additives with cryoprotectant effect. The method provides a faster, more effective and cheaper way to cryopreserve biological material.

### FIELD OF THE INVENTION

The present invention is related to the field of freeze preservation (cryopreservation) of cells, in particular vegetal cells including algae, organelles of (plant) cells and (plant) tissues.

### BACKGROUND OF THE INVENTION AND STATE OF THE ART

Plant cell and tissue cultures are important tools used extensively in both fundamental and applied research. They permit high-throughput studies such as gene functions and regulations, metabolic analyses or bio-pesticide discovery. They are also attractive and powerful bio-machineries to produce fine chemicals in bioreactors. Particularly, plant cell cultures are interesting to produce properly glycosylated and folded pharmaceutically active proteins (e.g. immunoglobulins, interleukins). They are intrinsically safe contrary to mammalian or microbial production platforms, because they neither host human pathogens nor produce endotoxins. Nevertheless, the maintenance of many wild type and transgenic cell lines in every laboratory dealing with plant biology is not only labor intensive and costly but also creates the risk of microbial contaminations, undesirable genetic changes (e.g., chromosomal modifications including variations in ploidy, epigenetic instability) and finally the loss of the culture.

In nature, the majority of animals and terrestrial plants use different mechanisms to enhance their survival under extreme environmental conditions. For instance plants can sense small temperature changes (1° C.) in order to adapt their growth and development. Besides, the majority of plants growing in temperate and cold climate zones are prone to freezing temperature during their life cycle. Under these environmental conditions, the major causes of cellular death are related to cellular dehydration and mechanical stress initiated by extracellular ice formation combined with severe damages of biological membranes enclosing organelles or the cell itself. Hence, plants have developed complex strategies to prevent cellular damages under mild freezing conditions. To survive, plants generally require a pre-exposure to non-lethal temperature, namely a cold acclimation step. This process involves multiple mechanisms including the accumulation of cryoprotective metabolites and antifreeze proteins. Because extreme temperature represents a significant stress for plants, freezing tolerance remains a critical factor limiting global plant distribution.

Integration of living cells within materials has been recognized as a powerful tool in biotechnology for over 50 years. Immobilization by adhesion on substrates, entrapment or encapsulation in materials are ways in which the benefits of whole cells can be harnessed in the construction of new devices such as bioreactors, biosensors, biofuel cells and artificial organs. As a matter of fact, cells isolated from their native environment are generally fragile. Cell-based biotechnological devices require therefore the integration of cells into abiotic materials.

Common strategies for the preservation of plant materials involve establishment of in vitro collection of plant tissues under growth conditions or colonies of plants in the field. For long term storage, these methods are not appropriate, because of concerns with cost and quality of the preserved plant tissues. Now, most plants cannot survive freezing and thawing cycles without high concentrations of cryoprotective agents (which can be cytotoxic) or dehydration procedures. Moreover, the success of these processes is closely linked to a complex cooling program. For instance, cells are cooled at an optimum rate to an intermediate temperature (slightly below the freezing point) and then vitrified in liquid nitrogen. In fact, these precautions permit to minimize cell dehydration and ice crystallization in the intracellular space, responsible for irreversible cellular damages. Finally, the biological materials thus obtained are stored within costly tank filled with liquid nitrogen in order to maintain a temperature of −196° C. Under these conditions, almost all metabolic activities cease and the living tissues can be preserved for extended periods of times.

At present, three different approaches have been proposed for the cryopreservation of plant cell cultures in liquid nitrogen:
(a) the slow cooling technique,
(b) the vitrification method and
(c) the dehydration of immobilized cells within alginate beads.

Various immobilizing or encapsulating matrices have been described. Far more sensitive than biomolecules, the immobilization of active cells involves the use of biocompatible materials, benign synthesis conditions and external fluidic support systems or immersion in buffer to avoid dehydration. The materials most often used are polysaccharides and derivatives, polymeric membranes, activated carbon as well as photopolymerized resins. However, these organic matrices exhibit several limitations in term of stability and biocompatibility.

Despite the widespread cryogenic storage of mammalian and microbial cultures, the limited reports of preservation methods used on plant cells are generally characterized by a low cellular viability.

In fact, plant cell suspensions, which consist of large vacuolated structures, are more prone to severe cryo-injury as compared to other cell types. The major causes of plant cell death are related to cellular dehydration and mechanical stress initiated by extracellular ice formation combined with severe damages to biological membranes enclosing organelles or the cell itself. Additionally, the current processes typically require costly liquid nitrogen, specialized apparatuses to provide a programmable cooling rate and high molarity of cryoprotectants which can be cytotoxic. For these reasons, alternative procedures are desirable for the creation of frozen master cell banks.

### AIMS OF THE INVENTION

The present invention aims to provide a method and kit for the freeze preservation or cryopreservation of (plant) cells, of (plant) cell organelles and (plant) tissues (comprising e.g., plant calluses, meristems, apices, shoot explants, ovules and roots) which do not present the drawbacks of the state of the art.

The present invention aims to provide such method and kit that is simple, less costly and which do not affect the characteristics of the treated (plant) cells, genetically modified (plant) cells, organelles of (plant) cells and (plant) tissues (including e.g., plant calluses, meristems, apices, shoot explants, ovules and roots) and which could therefore be used for the maintenance of cultures of (plant) cells, organelles of (plant) cells and (plant) tissues and keep their genetic characteristics over time, especially for a long period of several weeks or several months or even years.

### SUMMARY OF THE INVENTION

The present invention is related to a method for the the cryopreservation of a plant cell (including genetically modified plant cell), an organelle of this plant cell or a tissue of this plant which comprises (consists of) the steps of:
encapsulating this plant cell, cell organelle or this plant cell tissue within a porous silica matrix comprising one or more silica precursor(s) and one or more cryoprotectant(s);
incubating the obtained mixture at a temperature comprised between about 4° C. and about 20° C. for a period of more than 1 hour and transferring the resulting hybrid silica gels obtained in a freezer at a temperature comprised between −30° C. and −196° C., preferably at a temperature of about −80° C.

In the method of the invention, the encapsulating step is preferably obtained by mixing a solution comprising one or more silica precursor(s) and one or more cryoprotectant(s) with the plant cell, the plant cell organelle or the plant tissue to be encapsulated.

Another aspect of the present invention is related to a cryopreservation kit for (the cryopreservation) a genetically modified (plant cell), a plant cell organelle or a plant tissue comprising one or more silica precursor(s) and one or more cryoprotectant(s).

In the method and kit according to the invention, the cryoprotectant is preferably selected from the group consisting of Dimethyl sulfoxide (DMSO), a saccharide, an amino acid, a zwitterionic compound (betain), a glycol, polyol or a mixture thereof.

In the method and kit according to the invention, several different cryoprotectants can be mixed together for performing the method according to the invention.

Preferably, the saccharide is sucrose or trehalose, the amino-acid is preferably proline or glycine, the glycol is preferably (poly)ethylene glycol and the polyol is preferably selected from the group consisting of sorbitol, maltitol, glycerol, erythritol, xylitol, arabitol, mannitol, lactitol, isomaltitol or a mixture thereof.

In the method according to the invention, the concentration of silica precursors in the silica matrix can vary between about 5% and about 10%.

Preferably, the silica precursor(s) is (are) selected from the group consisting of polysilicic acid (H₂SiO₃)ₙ, preferably the metasilicic acid H₂SiO₃, a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium or potassium silicate), silica nanoparticules, sorbitylsilane, ormosils (organic modified silicas), trimethoxymethylsilane, dimethoxydimethylsilane, TMOS (tetramethoxysilane), DGS (diglycerylsilane) or a mixture thereof.

As a preferred embodiment in the method of the invention the cryoprotectants are sucrose and DMSO mixed with the silica precursor at a concentration comprises between about 0.2 M and about 1.0 M (for sucrose) and between about 1% and about 10% (DMSO) respectively

In the method according to the invention, the pH of the solution in the silica precursor and the cryoprotectant is preferably comprised between about 4 and about 8.

### DETAILED DESCRIPTION OF THE INVENTION

The present invention provides a simple, fast and cheap method for cryopreserving (plant, including algae) cells, genetically modified (plant) cells, organelles or tissue cultures. The technique is based on the protecting properties of silica matrices at low temperature (−30° C. or lower down to −196° C., preferably −70° C. or lower down to −196° C.) that can be used as an original, simple and efficient technique for the cryopreservation of (plant, including algae) cells, genetically modified (plant) cells, organelles or tissue lines in common laboratory freezers.

Silicon is naturally present in soil in the form of silicates (SiO₂·nH₂O). These mineral phases are weathered in water to produce monomeric orthosilicic acid (Si(OH)₄) and some disilicic acid which are taken up, transported and deposited as amorphous silica throughout the plant, particularly in the cell walls but not the vacuoles of the plant. Several reports highlighted that these Si species can improve plant resistance to bacterial and fungal attacks. Additionally, silica species alleviate a wide range of abiotic stresses, including nutrient imbalances, salinity, heavy metal toxicity, water stress, UV radiation, heat and freezing stress.

The claimed process of the invention requires neither specific apparatuses to control the cooling and/or warming rates, nor costly liquid nitrogen. The host structures maintain plant cells viability and do not impede their proliferation after a freeze-thaw cycle.

The main advantage of encapsulating (plant) cells within a silica matrix is that entrapped cells are more resistant to biotic (e.g. bacteria) or abiotic stresses (e.g. heat, water stress and heavy metals).

Because silica gel is a non-toxic compound compared to typical cryo-additives, cells can be successfully cryopreserved for short or long periods of time (longer than 2 years).

Based on this technique, the development of commercial plant-derived pharmaceuticals could become a more convenient approach according to recognized and well established guidelines.

Therefore, the present method is based upon the unexpected protecting effect of silica matrices at low temperature (at about −30° C. or lower, down to −196° C., preferably at about −70° C., down to −196° C.) which can be used as an original, simple and efficient technique for long-term cryopreservation of (plant, including algae) cells, genetically modified (plant) cells, (plant, including algae) cell lines, organelles (such as e.g., thylakoids) or tissues, in standard laboratory freezers, as well as in centers (such as the ATCC) dedicated for the conservation, especially cryopreservation, of cells and other biological materials.

A (plant) cell culture (such as, e.g., Arabidopsis thaliana) can be successfully cryopreserved by the method according to the invention, which comprises three essential steps.

The method of the invention comprises three essential and consecutive steps:

The first step comprises the encapsulation of (plant, including algae) cells, genetically modified (plant) cells, (plant, including algae) organelles (such as thylakoids) or (plant, including algae) tissues within a porous silica matrix via a sol-gel process. The obtained matrix, silica-based sol made of one or more silica precursor and one or more cryoprotectant(s). The silica precursor(s) is (are) chosen from the group consisting of a polysilicic acid (H₂SiO₃)ₙ (preferably metasilicic acid H₂SiO₃), a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium or potassium silicate), silica nanoparticules, sorbitylsilane, ormosils (organic modified silicas), trimethoxymethylsilane, dimethoxydimethylsilane, TMOS (tetramethoxysilane), DGS (diglycerylsilane), or a mixture thereof. More preferably, the silica precursor is the polysilicic acid (H₂SiO₃)ₙ, trimethoxymethylsilane, dimethoxydimethylsilane or a mixture thereof. Moreover, further additives, such as silica colloids (e.g., LUDOX®), silica co-precursors, or nanoparticles of silica can be added to the silica precursor solution.
Those additives function as additional sources of silica.
The cryoprotectant(s) is (are) preferably selected from the group consisting of DMSO (Dimethyl sulfoxide), an amino acid (such as proline or glycine), a zwitterionic compound (betaine) and a saccharide (trehalose, sucrose), a glycol (such as (poly)ethylene glycol or ethylene glycol) or a polyol (or polyalcohol, such as sorbitol, maltitol, glycerol, erythritol, xylitol, arabitol (lyxitol), mannitol, lactitol, isomaltitol, etc) or a mixture thereof.

The concentration of the silica precursors used can vary between about 5% and about 10% (WN). The type of silica precursors may influence the efficiency of the cryopreservation process.

Preferably, the cryoprotectant(s) is (are) mixed with the silica precursor at a concentration between about 1% and about 10% (DMSO) and between about 0.2 M and about 1.0 M (sucrose), respectively.

The second step (subsequent step) involves an incubation period of the obtained mixture at room temperature, preferably in a controlled room (at a temperature comprised between about 4° C. and about 20° C.) for a period of more than 1 hour, preferably from about 6 hours to about 48 hours or longer. The prepared hybrid gels are preferably kept in a closed flask.

The last step includes the transfer of the resulting hybrid silica gels in a laboratory freezer (at about −30° C. or lower, down to −196° C., preferably at about −70° C. down to −196° C.) without any specific precautionary measures.

The recovery of the (plant) cell suspension can be obtained by a quick warming of the sample vials at room temperature for a short time, for about 5 minutes to about 10 minutes. Thawed silica gels are then spread on a plate containing a solid nutrient medium. The host structures maintain (plant) cells viability and do not impede with their proliferation after a freeze-thaw cycle. After a period of about 7 days to about 14 days, recovered hybrid silica-cell materials are transferred into a flask containing fresh liquid medium. Alternatively, the cultivars are maintained on a solid nutrient medium. This method is effective, rapid and cheap and therefore, more efficient than current methods used, such as slow freezing techniques or dehydration of immobilized cells.

Another aspect of the present invention is related to a cryopreservation kit especially a cryopreservation kit for (plant, including algae) cells, genetically modified (plant) cells, (plant, including algae) organelles (such as thylakoids) or (plant, including algae) tissues that could be used in the method according to the invention and which comprises a silica matrix made of one or more silica precursor, such as polysilicic acid, and one or more cryoprotectant(s). The kit according to the invention can comprise these elements for the matrix according to the invention in separate vials. The silica precursor(s) is (are) preferably selected from the group consisting of a polysilicic acid (H₂SiO₃)ₙ (preferably metasilicic acid H₂SiO₃), a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium or potassium silicate), silica nanoparticules, sorbitylsilane, ormosils (organic modified silicas), trimethoxymethylsilane, dimethoxydimethylsilane, TMOS (tetramethoxysilane), DGS (diglycerylsilane), or a mixture thereof. The cryoprotectant(s) is (are) the one described above, such as DMSO (Dimethyl sulfoxide), saccharides (trehalose, sucrose), amino acids (proline or glycine) zwitterionic compounds (betaine), glycols, polyols (or polyalcohols, such as sorbitol, maltitol, glycerol, erythritol, xylitol, arabitol (lyxitol), mannitol, etc.) or a mixture thereof.

Preferably, the concentration of the silica precursors in the matrix can vary between about 5% and about 10% (WN). In a preferred embodiment, the cryoprotectant(s)(DMSO and Sucrose) is (are) mixed with the silica precursor at a concentration between about 1% and about 10% (DMSO) and between about 0.2 M and about 1.0 M (sucrose), respectively.

The present invention will be described in the following examples in reference to the enclosed figures as non-limiting preferred embodiments of the invention.

### SHORT DESCRIPTION OF THE DRAWINGS

FIG. 1 represents the determination of optimal conditions for the cryopreservation of A. thaliana cells via the monitoring of O₂-uptake (dark respiration) at 20° C. The metabolic activity of plant cells was evaluated after each of the three steps of the cryopreservation process. Effects of the incubation time of hybrid gels at different temperatures on cell activity after cryopreservation (A). Effects of sucrose (B) and DMSO (C) concentrations on cell preservation. 100% corresponds to the oxygen consumption of cells not having undergone any freeze-thaw cycle. The mean values (n=3) are presented with the standard deviations.

FIG. 2 represents the role of silica species in the cryopreservation of A. thaliana cells. Comparison of oxygen consumption of recovered cells that were pre-incubated with either polysilicic acid (A) or silica nanoparticles (B). The mean values (n=3) are presented with the standard deviations.

FIG. 3 represents the long-term preservation of plant cells. Effect of storage time of hybrid gels at −80° C. on post-thaw metabolic activity. The mean values (n=3) are presented with the standard deviations.

### EXAMPLES

### Example 1

### Optimal Conditions for the Cryopreservation of A. thaliana Cells

### Materials and Methods

Silica nanoparticles (5-15 nm), dipotassium tris(1,2-benzenediolato-O,O′)silicate, Murashige and Skoog medium (MSMO), sucrose, dimethyl sulfoxide (DMSO), potassium hydroxide, oxalic acid dehydrate 99%, ammonium molybdate tetrahydrate 99%, 4(methylamino)phenol sulfate 99%, sodium sulfite 99%, hydrochloric acid 37%, sulfuric acid 95%, kinetin and 1-naphtalenacetic acid were purchased from Sigma-Aldrich. Poly(Silicic acid)s (H₂SiO₃) was prepared from sodium silicate solution (Assay 25.5-28.5%, Merck) as described by C. F. Meunier, J. et al. (J. Mater. Chem., 2010, 20, 929-936). Amplex Red Hydrogen Peroxide assay kit and membrane filters of a pore size of 0.2 μm were obtained from Molecular Probes Co and Sartorius, respectively.

### Culture Conditions

Photomixotrophic suspension-cultured cells derived from the leaves of Arabidopsis thaliana strain L-MM1 ecotype Landberg erecta were cultivated in MSMO medium (4.4 g L⁻¹, pH 5.7) supplemented with 3% sucrose, 0.05 mg L⁻¹ of kinetin and 0.5 mg L⁻¹ of 1-naphthaleneacetic acid. Cells were maintained under 16/8 h light/dark photoperiod, at 22° C., on a rotary shaker at 115 round .per .minutes (r.p.m.)

### Cryopreservation of Cell Cultures

10 mL of cell suspensions (3 days after the latest subculture) were concentrated ten times in order to obtain a cell density of about 120 mg of fresh weight per milliliter. The plant cells were then transferred and mixed with 4 mL of a cryoprotecting silica sol (between 1 M and 3 M H₂SiO₃, between 1% and 10% (v/v) DMSO, between 0.2 M and 1 M Sucrose, about 4.4 g L¹ MSMO powder, pH value adjusted to between 4 and 8 with 0.2 M KOH) which was filter-sterilized with 0.20 μm membrane filters and refrigerated at 4° C. The mixture was then incubated in a cold room (4° C.) during about 1 to 6 hours. In the meantime, gelation occurred. The resulting hybrid silica gel was finally transferred and stored in a laboratory freezer (at about −80° C.).

### Thawing and Recovery

Hybrid silica matrices were quickly thawed by warming the sample vial at room temperature for about 5-10 minutes. Thawed gels were then spread on a plate containing a solid medium (0.8% agar, 4.4 g L⁻¹ MSMO medium supplemented with 3% sucrose, 0.05 mg L⁻¹ of kinetin and 0.5 mg L⁻¹ of 1-naphthaleneacetic acid) and incubated for about 7 days at 22° C. under 16/8 h light/dark photoperiod. After this period, recovered hybrid silica-cell materials were transferred into an Erlenmeyer flask containing 20 mL of fresh liquid medium.

### Post-Thaw Cell Viability.

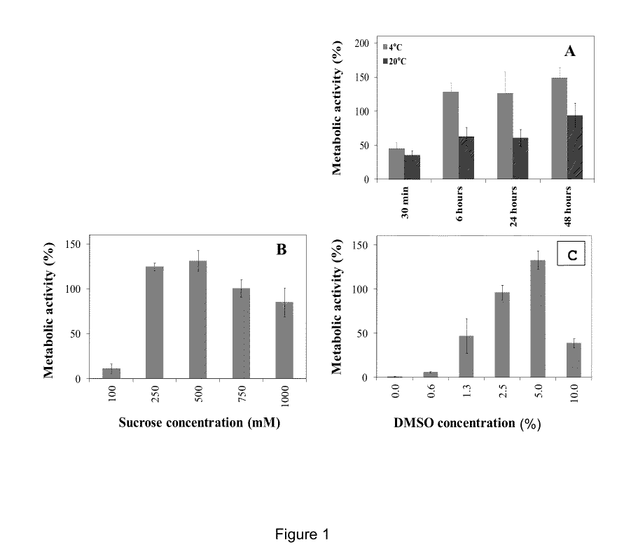

The physiological functions of cells was determined from about 1 hour to about 7 days after thawing by monitoring O₂-uptake at 20° C. with a Clark-type oxygen-electrode (Oxy-lab manufactured by Hansatech Instruments, UK). Cell viability was confirmed with a vital dye staining (fluorescein diacetate, FDA). Thawed cells were incubated with 5 mM FDA at room temperature for 5 minutes. Micrographs were collected at 536/40 nm with a color camera (DSRi1, Nikon) by illuminating the samples with a 482/35 nm excitation light using a fluorescent microscope (Multizoom AZ100 microscope purchased from Nikon). The ability of recovered cells to grow and form so-called callus tissues was also used as an indicator of cell viability.
The metabolic activity (monitoring O₂-uptake) of plant cells evaluated in after each of the three steps of the cryopreservation process: encapsulation (immobilization), incubation (4° C.) and freezing (−80° C.). In Figure A, the effect of the incubation step of hybrid gels at different temperatures (4° C. or 20° C.) was evaluated on cell activity after cryopreservation.
Then, the effects of sucrose (B) and DMSO (C) concentrations on cell preservation were evaluated. 100% corresponds to the oxygen consumption of cells not having undergone any freeze-thaw cycles.
FIG. 1A provides data on the optimum incubation time of the second step of process of the invention. As reported in FIG. 1A, the optimum incubation time varies from about 6 hours to about 48 hours.
FIG. 1B provides data on the optimum concentration in sucrose in the cryoprotecting silica solution of the first step of the process of the invention. As reported in FIG. 1B, the optimum concentration in sucrose varies from about 0.2 M to about 1 M.
FIG. 1C provides data on the optimum concentration in DMSO in the cryoprotecting silica solution of the first step of the process of the invention. As reported in FIG. 1C, the optimum concentration in DMSO varies from about 1% to 10%.

### Example 2

### Role of Silica Species for the Cryopreservation of A. thaliana Cells.

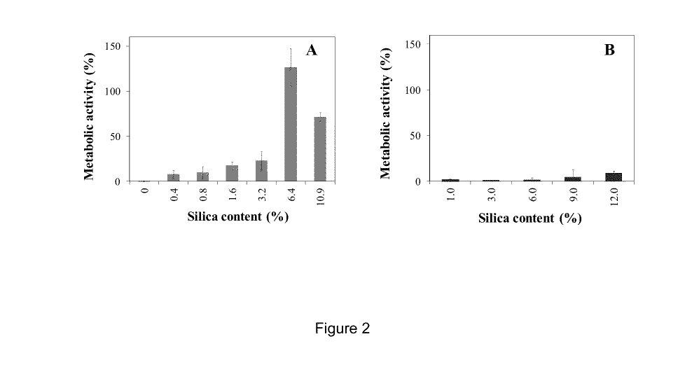

The experiment was performed as presented in example 1. Plant cells were transferred and mixed with 4 mL of a cryoprotecting silica sol containing different concentrations of polysilic acid (about 0.4% to about 10%), silica nanoparticles (about 1% to about 12%). FIG. 2 compares the oxygen consumption of recovered cells that were pre-incubated with either polysilicic acid (A) or silica nanoparticles (B).
As it can be derived from FIG. 2, polysilicic acid used as silica precursor provides a higher metabolic activity than silica nanoparticles within the concentration range tested.
As reported in FIG. 2A, the optimum concentration in polysilicic acid varies from about 5% to about 10%.
The optimum concentration in silica nanoparticles is of about 10%.

### Example 3

### Long Term Cryopreservation of A. thaliana Cells.

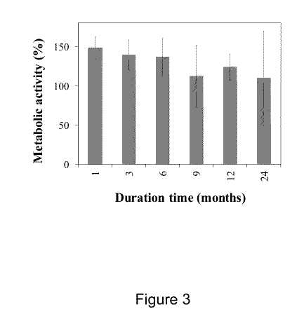

The experiment was performed as presented in example 1. FIG. 3 represents the oxygen consumption of recovered cells after storage at −80° C. for time varying from one month to 24 months.
Plant cells cryopreserved for two years within a silica matrix are still viable as reported in FIG. 3.

1. A method for cryopreservation of a plant cell, a plant cell organelle or plant tissue, which comprises the steps of:
encapsulating said plant cell, plant cell organelle or plant tissue within a porous silica matrix comprising one or more silica precursor(s) and one or more cryoprotectant(s);
incubating an obtained mixture at a temperature between 4° C. and 20° C. for a period of more than 1 hour; and
transferring resulting hybrid silica gels in a freezer at a temperature between −30° C. and −196° C.

2. The method according to claim 1, wherein the freezer is at a temperature of −80° C.

3. The method according to claims 1, wherein the cryoprotectant(s) is (are) selected from the group consisting of dimethyl sulfoxide (DMSO), a saccharide, an amino acid, a zwitterionic compound (betaine), a glycol, a polyol or a mixture thereof.

4. The method according to claim 3, wherein the saccharide is sucrose or trehalose.

5. The method of claim 3, wherein the amino acid is proline or glycine.

6. The method of claim 3, wherein the glycol is (poly) ethylene glycol.

7. The method according to claim 3, wherein the polyol is selected from the group consisting of sorbitol, maltitol, glycerol, erythritol, xylitol, arabitol, mannitol, lactitol, isomaltitol or a mixture thereof.

8. The method according to claim 1, wherein the concentration of silica precursors in the silica matrix can vary between 5% and 10%.

9. The method according to claim 1, wherein the silica precursor(s) is (are) selected from the group consisting of a polysilicic acid (H2SiO3)n (preferably metasilicic acid H2SiO3), a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium or potassium silicate), silica nanoparticules, sorbitylsilane, ormosils (organic modified silicas), trimethoxymethylsilane, dimethoxydimethylsilane, TMOS (tetramethoxysilane), DGS (diglycerylsilane), or a mixture thereof.

10. The method according to claim 1, wherein the cryoprotectants are sucrose and DMSO mixed with the silica precursor(s) at a concentration comprised between 0.2 M and 1.0 M (sucrose) and between 1% and 10% (DMSO), respectively.

11. A cryopreservation kit for a plant cell, a plant cell organelle or a plant tissue comprising one or more silica precursors and one or more cryoprotectant(s).

12. The kit according to claim 11, wherein the silica precursor(s) is (are) selected from the group consisting of a polysilicic acid (H2SiO3)n (preferably metasilicic acid H2SiO3), a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium or potassium silicate), silica nanoparticules, sorbitylsilane, ormosils (organic modified silicas), trimethoxymethylsilane, dimethoxydimethylsilane, TMOS (tetramethoxysilane), DGS (diglycerylsilane), or a mixture thereof.

13. The kit according to claim 11, wherein the cryoprotectant(s) is (are) selected from the group consisting of DMSO (Dimethyl sulfoxide), a saccharide, an amino acid, a zwitterionic compound (betaine), a glycol or a polyol or a mixture thereof.

14. The kit according to claim 13, wherein the saccharide is sucrose or trehalose.

15. The kit according to claim 13, wherein the amino acid is proline or glycine.

16. The kit according to claim 13, wherein the glycol is (poly) ethylene glycol.

17. The kit according to claim 13, wherein the polyol is selected from the group consisting of sorbitol, maltitol, glycerol, erythritol, xylitol, arabitol, mannitol, lactitol, isomaltitol or a mixture thereof.
